(19) United States
(12) Patent Application Publication
(10) Pub. No.: US 2014/0135834 A1
(43) Pub. Date: May 15, 2014

(54) KNOTLESS SUTURE ANCHORS WITH RATCHET MECHANISM

(71) Applicant: Kevin A. Mansmann, Paoli, PA (US)

(72) Inventor: Kevin A. Mansmann, Paoli, PA (US)

(21) Appl. No.: 13/673,081

(22) Filed: Nov. 9, 2012

(51) Int. Cl. A61B 17/04

(52) U.S. Cl. CPC A61B 17/0401
USPC 606/232

(57) ABSTRACT

A device for knotless securing of surgical sutures has a component such as a tissue embeddable anchor, carrying a mechanism that permits a suture to be pulled through the device in one direction but prevents travel in the opposite direction. The suture passes through the device between an entry and exit. A movable brake in the anchor or other component interacts with the suture. Tension on the suture in one direction pinches the suture by pulling the brake against the suture at a seating surface, preventing movement of the suture in that direction. Tension in the other direction lifts the brake and frees the suture.

### BACKGROUND

This invention is in the field of surgery, and relates to “suture anchors” and similar devices that are used to handle, tighten, and secure suture strands, cerclage wires, and other stranded and/or fibrous devices or components, especially in locations that are difficult for a surgeon to reach and manipulate. These devices are especially useful for “minimally invasive” surgery, such as arthroscopic or laparoscopic surgery, and for attaching suture strands and cerclage wires to both hard bones, and soft tissues.

As used herein, the term surgery and its related terms (such as surgical) refer to controlled manipulation and alteration of cohesive tissue, by a qualified and licensed physician. In all cases of interest herein, surgery requires and involves some form of penetration of soft tissue, in ways that will require at least one incision and/or controlled cutting step. It does not cover or include: (i) injections of cell suspensions or other fluids into tissues, if such injections do not also involve at least one substantial cut or incision; or, (ii) manipulations or alterations that are limited to surface-accessible skin or mucous membranes (such as dermal abrasion, or similar surface-only cosmetic-type procedures).

Various efforts have been made to design and create surgical devices called “knotless suture anchors”, for use in handling, tightening, aid securing suture strands during various types of surgery. Whenever surgery is being performed, efforts are made to minimize the number and the lengths or sizes of any punctures or incisions that must be made through the skin, or through “sub-dermal” (i.e., below-the-skin) tissues. Such efforts help minimize pain, discomfort, and scarring, and they also promote improved long-term recovery, repair, and restructuring of the surgically-altered tissues, and of any overlaying and surrounding tissues that must be penetrated in order for the surgeon to reach the injured or diseased tissue(s) that require repair. Accordingly, nearly any type of surgery can be regarded as“minimally invasive” under a broad interpretation of that phrase.

However, a specialized type of surgery which truly can be regarded as “minimally invasive” has been developed over the past few decades. It is exemplified by: (1) arthroscopic surgery (i.e., surgery on articulating joints, such as knees, shoulders, etc.); and, (2) laparoscopic surgery (i.e., surgery on the abdominal organs, which are surrounded by membranes and muscles that must be penetrated to reach those organs). Both of those specialized types of surgery use long and slender instruments that are designed to penetrate, to any desired depth, through small skin incisions, which typically are limited to only about 1 cm (about half an inch) in length, whenever possible.

The challenges of handling (and tying knots in) suture strands, during arthroscopic or laparoscopic surgery, become especially complex when one realizes that either such type of surgery usually requires, in addition to the actual surgical instruments, a number of supporting devices, which in most cases will include: (i) a light source; (ii) a camera with a live video feed, which normally will use fiber-optic cables; (iii) a tube which will continuously pump clear saline liquid into the operating field, to carry blood and debris out of area so that the surgeon can see the structures and tissues that are being manipulated; and, (iv) a drainage catheter or cannula, to suction the saline liquid and its contents out of the joint or body cavity.

In addition, surgeons are under pressure to work as quickly and efficiently as possible, starting when a patient is placed under anesthesia, and lasting until the patient's joint, limb, or body has been closed up and covered with bandages. As a general principle, the longer a patient's body or limb stays open, the greater will be the risk, threat, and likelihood of infection. In addition, if a patient must stay under anesthesia for an extended period of time, the risks increase for potentially serious disruptions to his or her nervous or autonomic system.

Under those conditions and pressures, the challenge of tying a knot in a suture strand, especially in a location that may be on the far (distal) side of a bone or other anatomic structure, using only one or possibly two elongated instruments comparable to needle-nose pliers, can be very difficult.

Just as importantly, with regard to this current invention, once a knot has been tied, it usually is very difficult to subsequently adjust the tension (i.e., the level of tensile force, which can also be called tightness, tautness, or similar terms) in that suture strand. However, as described below, there are numerous situations in which a surgeon may wish to adjust and tighten a plurality of strands in a stepwise, coordinated manner.

As one example, if a cartilage-replacing implant device is being affixed to a bone in a load-bearing joint, such as a knee, a preferred installation procedure is likely to involve taking a number of reinforcing suture strands through a procedure referred to herein as, “start them all, then snug them all, then tighten them all” (also referred to simply as a “start-snug-tighten” procedure, for brevity). As described in more detail in U.S. patent application Ser. No. 13/355,276 (by the same Applicant herein), this type of approach is analogous to the way a set of lug nuts can and should be taken through a similar set of “start them all, then snug them all, then tighten them all” steps, when replacing a flat tire on a car or truck.

As another example, when a rotator cuff in a shoulder joint becomes damaged and torn, the damage usually involves detachment of certain tendons and ligaments from the humerus (i.e., the long bone in the upper arm). When that type of damage occurs, the torn tendons and ligaments tend to retract deeper into the shoulder joint, generally toward a patient's shoulder blade. Accordingly, a typical rotator cuff repair requires a surgeon to gently but firmly pull the torn tendon and ligament segments back in an “outward” direction, so that they can be reattached to the enlarged bone structure at the upper end of the humerus (its full technical name is “the greater tuberosity of the humerus”; it is also called “the humeral head”). Once those damaged tendon and ligament segments have been pulled in an outward direction, back into their proper position, the surgeon does his best to reattach them to the “head” of the humerus. That surgical process, of pulling and tugging damaged soft tissues closer to their original position and then attaching them to a bone or other structure, is often referred to as “approximating” the damaged tissue.

If a surgeon attempted to perform that type of procedure on a torn rotator cuff, using only a single suture strand which has been passed through a single needle track (which requires a new and fresh puncture wound, through the tissue that needs to be pulled back into place), then two adverse results are likely:

(1) the high levels of stress that are imposed on the damaged tissue, at the new puncture wound, will create a seriously increased risk of tearing and damaging the already-damaged tissue, even more; and,

(2) the success and accuracy of the “approximation” procedure would be seriously compromised, and degraded. It would be comparable to try to make up a bed, to get it to look nice, while being allowed to grip a bed cover at only a single location, using only one finger and the thumb to grip the sheet at that point.

Accordingly, in a better approach, the “leading edge” of a torn rotator cuff usually has several suture strands attached to it, at spaced locations along the “leading edge”, and the surgeon does his/her best to use all of those suture strands to gently pull, tug, and coax the edge of the rotator cuff back into its properly position along that entire edge, before taking steps to secure it to the bone along that edge, which will remain under tension, during the reattachment procedure. A typical “single row” approximation usually involved at least 2, and up to 4, suture strands, laced through a series of spaced locations along the edge of the rotator cuff tissue. Many surgeons use a “double row” approach, with twice as many suture strands, and some surgeons will even use a “triple row” approach, if a rotator cuff is badly damaged and requires unusually high levels of distributed pulling force, and post-surgical reinforcement.

That type of surgical “approximation” and reattachment can be substantially improved (i.e., it can be performed more rapidly and easily, and with better long-term results), if a surgeon can use an entire set of ratcheting knotless suture anchors (i.e., one for each suture strand, while using multiple suture strands), during that type of surgery.

In some cases, surgical staples can be well-suited, for securing soft tissues to other soft tissues. However, they usually are not well-suited for securing soft tissues (or suture strands which have been attached to soft tissues) to hard bone surfaces. When attachments to hard bone are required, devices that are stronger and more secure (and which are usually called anchors, rather than staples) are used. Some are designed to be screwed or tapped into a “pilot hole” that has been drilled into a bone; others are driven directly into a bone surface, in a manner comparable to driving a nail into a board with no pilot hole.

Accordingly, surgeons and orthopedic supply companies have developed various types of “knotless suture anchors”, which enable surgeons to attach suture strands (which, in most cases, will be attached to soft tissues, and which can provide convenient “handles” that can be used to pull, stretch, pin down, or otherwise manipulate the soft tissues) to hard bone surfaces. These types of knotless suture anchors are described and illustrated in a number of issued patents and published patent applications.

The types of “knotless suture anchors” that are taught in the prior art can be divided into several categories, for purposes of analyzing and understanding them.

A first category involves anchors that will undergo some type of shape alteration, after they have been inserted into a drilled hole, in a manner which will cause a set of projections to extend outwardly from the main body of the anchor. The projections will press against or dig into the walls of the pilot hole in the bone, thereby firmly securing the anchor to the bone and preventing it from being pulled out by any tensile forces which are likely to be imposed on the suture strand. On some of these types of anchors, the projections have spring-type or angled structures that are similar to the “barbs” on a harpoon or fishing hook; on other anchors in this category, the projections are more closely comparable to the types of “expander bolts” that are used to mount large paintings and other heavy wall-hangings to drywall or sheetrock, in homes and other buildings. Issued patents which describe these types of suture anchors include, for example, U.S. Pat. No. 6,328,758 (Tornier et al 2001), U.S. Pat. No. 7,144,415 (Del Rio et al 2006), and U.S. Pat. Nos. 7,556,640 and 7,695,494 (both by Foerster et al, 2009 and 2010).

A second category of knotless suture anchors include two components which are separate from each other before installation. Because of how they interact and function, the two components can be regarded as a receptacle, and an insert. In this type of design, the receptacle component is implanted into a bone, normally into a pilot hole. After the receptacle component has been fully inserted into the bone, the insert component is inserted into the receptacle, typically using tapping, screwing, or similar efforts to drive the insert far enough into the receptacle to lock them together. In some of these anchors, the receptacle component will be fully anchored to the bone, before the insert component is emplaced in the receptacle; in other designs, the act of forcing the insert into the receptacle will cause a shape change which completes the anchoring of the receptacle to the bone.

In these types of knotless suture anchors, a suture strand typically is looped around, passed through, or otherwise coupled or affixed to the insert component, before the insert component is inserted into the receptacle. In some designs, the act of driving the insert into the receptacle will squeeze, crimp, or otherwise secure the suture strand to the anchor, in a manner which cannot be altered later without difficulty; an example of this type of design is provided by U.S. Pat. No. 7,572,283 (Meridew 2009). In other designs, a yielding elastomeric fit between the insert and the receptacle will allow subsequent adjustments to the suture strand, if a tensile force is exerted on the strand which exceeds some type of “threshold” force level; this design is illustrated in several published applications by McDevitt et al, such as US 20030130695. Still other designs enable the insert component to be manipulated in a way that will allow the receptacle component to be removed from the bone, if needed, in case the tension on the suture strand which is held by that anchor needs to be adjusted after an initial fixation; this type of design is illustrated by U.S. Pat. No. 6,540,770 (Tornier et al 2003).

Other designs for knotless suture anchors with various other traits are provided by a number of other issued patents and published applications. These include, for example, U.S. Pat. No. 6,520,980; U.S. Pat. No. 6,585,730; U.S. Pat. No. 7,682,374; and U.S. Pat. No. 7,637,926, all issued to Foerster et al and assigned to either Opus Medical Inc. or ArthroCare Corporation.

A different type of design, which involves a ratcheting suture anchor, is described and illustrated in two published patent applications, US 2010/0063542 and 2010/0121348, both by Van Der Burg et al. In this design, a suture strand is wrapped around an internal component which can rotate, in a ratcheting manner, within an outer sleeve component. The ratcheting mechanism is provided by a pin, affixed to the top of the rotating internal component, which travels along a sawtooth surface provided by the outer sleeve. The pin can “ride up” each sloped incline on the sawtooth surface. Each time it reaches the top of an incline, it will drop down a steep edge, into a settling location. This effectively prevents the ratchet mechanism from traveling in the non-allowed direction, unless the surgeon takes special steps to disengage the pin from the sawtooth surface so that the tension on the suture strand can be adjusted.

Finally, the closest known prior art is a type of suture anchor being sold by the Tornier company (their US affiliate has a website at www.tornier-us.com), under the trademark PITON. It is described and illustrated at http://www.tornier-us.com/sportsmed/smd003/piton35.php?pop=1 and other pages on that website, and it is believed to be covered by U.S. patent application Ser. No. 13/073,342, which was published as 20110238113 (Fanton et al, filed on Mar. 28, 2011). Briefly, that type of anchor has a pointed shell which can be driven into a bone surface, and two metallic loop-type components, both of which are movable, which function in a manner that is comparable to the type of double-ring structure commonly known as a “cinch buckle”.

As described below, one of the distinctions between the invention disclosed in this current application, and the type of “cinch” mechanism used in the Tornier PITON anchors, is that the rachet mechanism in the current invention has only a single moving part. As described below, that difference can lead to potentially important advantages, especially in certain types of orthopedic surgery.

### Ratchet (Also Spelled Rachet) Mechanisms

Some sources assert that “ratchet” is the proper spelling for the types of devices and mechanisms discussed herein; however, other sources assert that “rachet” is the proper spelling. Accordingly, both spellings should be regarded and accepted as alternate correct spellings.

In addition to having two different spellings, the term “ratchet” has acquired several different meanings, and shadings. Those different meanings can lead to confusion and conflict, if not understood. These will be briefly addressed herein, in order to clarify and establish the definition of an important term used herein.

A strict and narrow definition of “ratchet” (which is pointed out herein, for informational purposes, but which is not adopted and used herein) requires the presence of both:

(1) at least one gear, with some type of teeth or projections (this gear can be either a “round” or “star” type gear, or a linear-type gear, as found in “rack and pinion” devices, and in so-called “cable ties”); and,

(2) a “pawl” mechanism which either:

(a) allows the gear to rotate in only one direction; or,

(b) allows some type of assembly to travel along a linear or comparable gear in only one direction.

An unstated but implied qualifier, in any and all statements herein which refer to prevention of travel in a prohibited or impeded direction, can be stated as, “unless and until the ratchet mechanism is disengaged.” As anyone who understands ratchet mechanisms will recognize, if a pawl component is disengaged from a gear surface, or if other steps are taken to dismount (or disengage, deactivate, etc.) a ratchet mechanism (such as by pulling a braided rope out of and away from a “cam cleat” mechanism), then the “directional travel” prohibition or impedance is removed, and travel in that otherwise blocked direction will no longer be blocked by the ratchet mechanism.

Classic ratchet systems, with round (or star) rotating gears, and with pawl components that allow the gear to rotate in only one direction, are illustrated in any basic introduction to ratchet mechanisms, such as FIGS. 1 and 2 of the Wikipedia entry on “ratchet mechanisms”. A similar drawing, with callout numbers used to identify the various components of a rotating-gear ratchet system, is provided in FIG. 1 of U.S. patent application Ser. No. 13/355,276, by the same Applicant herein.

The Ser. No. 13/355,276 application is entirely devoted to various types of ratcheting knotless suture anchors. Accordingly, everything set forth in that application is relevant, herein. That application describes and illustrates a number of candidate ratchet mechanisms that can be miniaturized and adapted for use as ratcheting mechanisms within knotless suture anchors. Subsequently, after that application was filed, the Applicant herein (the same applicant in the '276 application) conceived of a new and different internal mechanism for ratcheting knotless suture anchors. Because that new and additional mechanism appears to be ideally suited for use in knotless suture anchors, this additional and supplementary application is being filed, to cover that new design and mechanism.

Accordingly, U.S. patent application Ser. No. 13/355,276 describes and illustrates several mechanisms that are classified as “ratchet” mechanisms, even though they do not have a classic “gear and pawl” interaction of a type that is required by a strict and narrow definition of “ratchet”. Those other mechanisms include:

(1) non-round “cam cleats” with ridged, saw-toothed, or other non-smooth surfaces, which use cam-type mechanisms to exert a stronger and tighter grip on a rope, cable, or other tensile member, if and when the rope, cable, or other tensile member attempts to travel in the prohibited direction;

(2) a sleeve-type enclosing surface that is provided with numerous stiff and angled (or “biased”) bristles, which will allow a braided strand to slide through the bristles in one direction, but which will quickly begin to poke into and settle into the interstitial spaces in a braid strand, if the braided strand begins to travel in a prohibited direction;

(3) a suture anchor with a crimping mechanism, which can be driven into a bone or other tissue in a relatively straight orientation, and which will begin to bend and distort, around a hinge-type component, in a manner which will pinch and crimp the suture strand, as the suture strand is pulled in a desired direction.

Those types of ratcheting mechanisms fall within a definition of “ratchet” that is broader and more widely used than the strict “gear-and-pawl” definition of “ratchet”. Under this broader definition, a device or mechanism qualifies as a “ratchet” mechanism (or a “ratchet-type mechanism”), if it:

(i) allows a certain component (which can be a tensile (i.e., tension-bearing) member, such as a rope, string, cord, belt, chain, cable, or suture strand) to travel in a first allowed direction, without a level of resistance which would impede normal motion or travel of the ratchet mechanism in the allowed direction; and,

(ii) prevents that same component from traveling in the opposite direction, with a degree of security and reliability that is high enough to carry out and withstand reasonable and anticipated uses of that particular type of ratchet mechanism.

That definition is stated in that type of functional manner (i.e., referring to factors such as “reasonable and anticipated uses” of a ratchet mechanism), because of two limiting factors that must be recognized and understood.

The first limiting factor is this: a moment of contemplation will let anyone realize that any ratchet mechanism can fail, and will thereafter allow travel in the normally prohibited direction, if the ratchet mechanism is subjected to forces or conditions that are strong enough and severe enough to deform and damage the mechanism. Indeed, that caveat applies to any mechanism or device ever built by humans; ANY mechanism can fail, if subjected to forces strong enough to deform and damage the mechanism. Accordingly, the fact that some particular ratchet mechanism can be damaged to a point of failure, and may allow travel in an unwanted direction after it has failed, does not prevent such mechanisms from falling within the definition of “ratchet”, based on their design and on their normal mode of functioning.

The second limiting factor reflects the fact that various types of mechanisms fall into a zone of uncertainty, where it is not clear whether they do or do not properly and accurately qualify as “ratchet” devices or system. This group of devices is illustrated and exemplified by the type of belt buckle often called a “cinch buckle”. This type of buckle, which is often found on woven or braided belts that are used to hold up trousers (cinch buckles normally are not used with leather belts or straps, since they would damage the leather), involves two metallic rings which are adjacent or close to each other, where they effectively become “parallel” circles or arcs. Each metal ring will have a portion (which can be a straight segment, within an otherwise circular ring) that is constrained within the webbing or fabric of the belt. When the free end of a belt is looped through a “cinch buckle”, the act of looping the belt over and around the “top” ring, before lacing it back through the lower ring and then pulling it tight (so that the rough or textured surface of a woven or braided belt will be pressed against itself) creates a squeezing and crimping force which pulls and presses the upper ring (and its loop of belt material) downward against the lower ring. In this manner, the two adjacent metal rings can squeeze and effectively grab a woven or braided belt, with sufficient strength to allow the belt to function adequately, in holding up trousers.

Accordingly, a cinch buckle can qualify as a ratcheting device, under a broad definition of “ratchet”, since it allows one end of a belt or strap to be pulled in one direction (i.e., in a tightening direction), and it then generally prevents that end of the belt or strap from traveling in the opposite direction (which would quickly loosen the belt or strap).

However, it must be recognized that a cinch buckle can only generally prevent (or hinder, impede, or similar terms) the travel of a belt or strap in a non-allowed direction. A cinch buckle does not have any mechanism which truly prevents and prohibits small amounts of travel in an unwanted direction (small amounts of travel are often referred to as slippage, creep, etc.). In general, a belt with a cinch buckle is adequate for holding up trousers, only if the person wearing the belt is able to conveniently and discretely reach down and tighten the belt whenever necessary, during the course of a day or evening each time the belt becomes too loose to function effectively. If desired, the surfaces of the rings can be have knurled or other rough or textured surfaces, which can help reduce slippage, but those types of steps do not change the nature of a cinch buckle.

To a large extent, the proper use of terms such as “ratchet” will depend on the setting, functional requirements, and context of the usage. For example, a cinch buckle might properly and reasonably be referred to as a ratchet mechanism, if used to secure a belt around a duffel bag or comparable item that is being used to store or transport clothes or other lightweight items. However, a cinch buckle cannot be used to safely secure heavy cargo to a flatbed trailer, in the types of 18-wheeler trucks that haul cargo across highways. Since the risk of a cinch buckle gradually losing its “grip” on a strap or belt is so high, in an environment where vibration, jostling, or other repetitive motion occurs (and where unintended release of the cargo, from a truck driving at high speed down a highway, might kill or maim innocent people), it would constitute reckless disregard and even criminal neglect if a trucking company used nothing but “cinch buckles” on the straps they use to secure heavy cargo to truck trailers. Accordingly, in that type of setting, a cinch buckle should not be referred to as a ratchet mechanism.

Therefore, rather than relying solely and strictly on mechanical terms or limits, a more comprehensive and insightful definition of “ratchet mechanism” (or rachet-type mechanisms, as used in the claims) should recognize that it includes mechanisms which will do both of the following:

(i) allow a tensile member to travel in a first allowed direction, with only a minimal or modest level of resistance and hindrance, which can be overcome in a relatively simple and non-destructive manner; and,

(ii) prevent and prohibit that same tensile member from traveling in the opposite direction, with a degree of security and reliability that is high enough to carry out and withstand reasonable and anticipated uses of that particular type of ratchet mechanism.

With regard to the type of “ratcheting knotless suture anchors” that are disclosed and described herein, these types of mechanisms have been fabricated in prototype form, and have received initial testing. The results have indicated that these types of ratchet mechanisms are indeed well-suited for surgical use as described herein, and will adequately prevent and prohibit (rather than merely impede or hinder) travel of a suture strand in an unwanted direction, in a manner and with a level of gripping strength and security which will enable a surgeon to establish a desired level of tension in each of numerous suture strands that are being used to “approximate” and reattach damaged tissue (or to implant a device that will permanently replace damaged tissue, such as a segment of cartilage), in a manner which allows the surgeon to:

(i) create a desired level of tightness (or tautness, tensile force, etc.) on each suture strand, among a plurality of suture strands that are being used to manipulate an extended edge or area of damaged tissue (or along an edge or perimeter of an implant device), in a manner which establish a desired level of tensile force in each strand, while all of the previously-tightened strands remain tight and generate an overall pulling force that is distributed in a relatively even manner across an edge or area of damaged tissue (or across an edge or perimeter of an implant device); and,

(ii) return to any previously-tightened suture strand, as many times as desired, and quickly and efficiently increase the level of tightness in each strand, using as many “tensioning cycles” as desired, to exert a distributed, controlled, and effective pulling force, such as (i) on a relatively long or large implant device; and/or, (i) across a relatively large edge or area of tissue.

Stated in other words, each suture strand, within an entire set of suture strands, is provided with its own convenient and easily-operated handling and anchoring device. Each such anchor contains a miniaturized ratchet mechanism, which allows a free end of the suture strand to pass through the ratcheting mechanism, so that the free end of the strand can be gripped directly, by a surgeon's tool, and pulled to any desired level of tightness (i.e. tensile force), during each step in a multi-step procedure that uses multiple strands, spaced apart from each other by distances that are chosen and established by a surgeon, to exert a distributed pulling force on an edge or area of tissue, or on an implant device which is being positioned on and then secured to a bone or other tissue.

In general, any skilled surgeon who has used the types of ratcheting suture anchors described herein can determine, for any individual patient, whether additional steps should be taken to supplement the “grip” which will be provided by these suture anchors, over a span of time that may be measured in weeks or months, in locations such as joints that may be subjected to repeated, cyclic, and/or intermittent stresses or movement. If a surgeon chooses to do so, supplemental steps such as the following can be taken:

(1) the free end of the suture strand can be wrapped once or twice around the outer barrel or sleeve of the anchor (preferably while inserting the free end of the strand beneath the “incoming” segment of suture strand, to prevent any risk of the strand slipping off of the anchoring device). The strand can then be tied in a conventional knot, in a manner that will affix it to the anchoring device with no risk of subsequent slippage (or “creep”) of the suture strand through the ratchet mechanism in the prohibited direction; and/or,

(2) the free end of a suture strand can be affixed to a bone screw, soft tissue, or other attachment point, using conventional means, while the ratcheting anchor device continues to grip and hold the strand at a “midpoint” location.

Accordingly, one object of this invention is to disclose certain specific types of ratcheting mechanisms which can be utilized in knotless suture anchors, which will provide sufficiently high levels of security, stability, and reliability to justify and enable their use in various types of surgery where racheting knotless suture anchors can be valuable and helpful.

Another object of this invention is to disclose and provide methods for improved anchoring of surgical implants that contain tissue-ingrowth surfaces, using a combination of: (i) suture segments which emerge from such implants, and (ii) knotless ratcheting-type suture anchors as described herein, which are designed to allow surgeons to tension and tighten, in a staged, sequential, and controlled manner, each of the suture segments that emerge from various locations around the periphery of an implant.

Another object of this invention is to disclose designs which are believed to be novel, for knotless suture anchors that have ratcheting mechanisms which will allow surgeons to tension and tighten, in a staged, sequential, and controlled manner, each of a number of suture segments that will be used to either: (1) pull a segment, edge, or area of damaged tissue into a desired position for repair purposes, using multiple tensile elements to distribute a pulling force and minimize any potentially destructive tearing forces; or, (2) anchor a surgical implant device with greater levels of strength and stability.

These and other objects of the invention will become more apparent through the following summary, drawings, and detailed description.

### SUMMARY OF THE INVENTION

Miniaturized devices are disclosed that will provide ratcheting knotless anchors for use in combination with suture strands, for use during various types of human or veterinary surgery. These knotless suture anchors will contain one or more types of ratcheting mechanisms, to allow a surgeon to pull a suture strand through an anchor device in one direction, without allowing the suture strand to travel or creep backward, in the other direction. This mechanism comprises a movable “bead” or “brake” component, which will either:

(1) press against an accommodating seating component, in a manner which will pinch and grip the bead or brake component, if the suture strand is pulled in a travel-prohibited direction; or,

(2) press against a non-pinching, non-gripping surface, in a manner that allows sliding-type travel of the suture strand, when a surgeon pulls the strand through the suture anchor in a travel-allowed direction.

In a preferred embodiment, the movable bead component has a hole (or tunnel, conduit, etc.) passing through it. Any suture strand(s) which is/are handled by that suture anchor are passed a first time through that hole, then wrapped halfway around the bead, then passed through that same hole a second time. This will cause any travel or pulling of the suture strand, in either direction, to carry the bead and cause it to press against either: (i) the seating component, which will grip the suture strand in a manner which prevents additional travel; or, (ii) a non-pinching exit component, in a manner which allows a surgeon to pull the suture strand through the suture anchor, in that direction.

Unlike a rotating ratchet mechanism which must be driven by some type of wrench or other tool, the ratchet mechanism disclosed herein allows a suture strand to pass entirely through the ratcheting knotless anchor, in a manner that will provide a “free end” of the suture strand which can be grasped by a surgeon (normally with the aid of minimally-invasive tools, comparable to needle-nose pliers) and pulled to any desired level of tension and tightness. This can preserve a normal and natural “feel”, which will allow a surgeon to directly feel and monitor the progress of the tightening steps for any suture.

### BRIEF DESCRIPTION OF THE DRAWINGS

FIG. 1 is a side (elevation) view, with a partial cutaway, showing another type of ratcheting knotless suture anchor (also shown in FIG. 2) having a movable “brake” (or bead, etc.) with a suture strand wrapped around it. When a surgeon pulls the free end of the suture strand (i.e., from the right side of device shown in FIGS. 19 and 20), the suture strand will be pulled through the bead, with a sliding nation. When the surgeon releases the suture strand, the tension exerted on it by the implant device will pull the movable brake/bead against a “seating component” having a conforming shape. This will pinch and effectively trap the suture strand, to prevent it from being pulled toward the implant.

FIG. 2 is a top (plan) view of the ratcheting suture anchor shown in FIG. 1.

FIG. 3 (which includes panels 3A and 3B) depicts a somewhat different design for a ratcheting suture anchor having a movable brake component which is slidably coupled to a suture strand. As with the device shown in FIGS. 1 and 2, this device will: (i) allow a braided suture strand to pass through the anchor in an allowed direction, with little resistance; and, (ii) create a pinching, gripping, or crimping action, which will trap the suture strand between the brake component and a seating surface, when a tensile force attempts to pull the strand in the non-allowed direction.

FIG. 4 depicts a ratcheting knotless suture anchor that is assembled in situ, during surgery, comprising: (1) a lower anchoring component, surrounded by a flexible apron-type component that can be anchored to soft tissue by means of sutures, staples, etc., before the upper component is inserted into the patient's joint or body; and, (ii) an upper component, which is designed to be attached to an already-anchored lower component, and which will contain a movable-brake type of ratchet mechanism as described herein.

FIG. 5 depicts a surgical kit, with various implantable components inside a sealed envelope which ensures their sterility until use by a surgeon.

### DETAILED DESCRIPTION

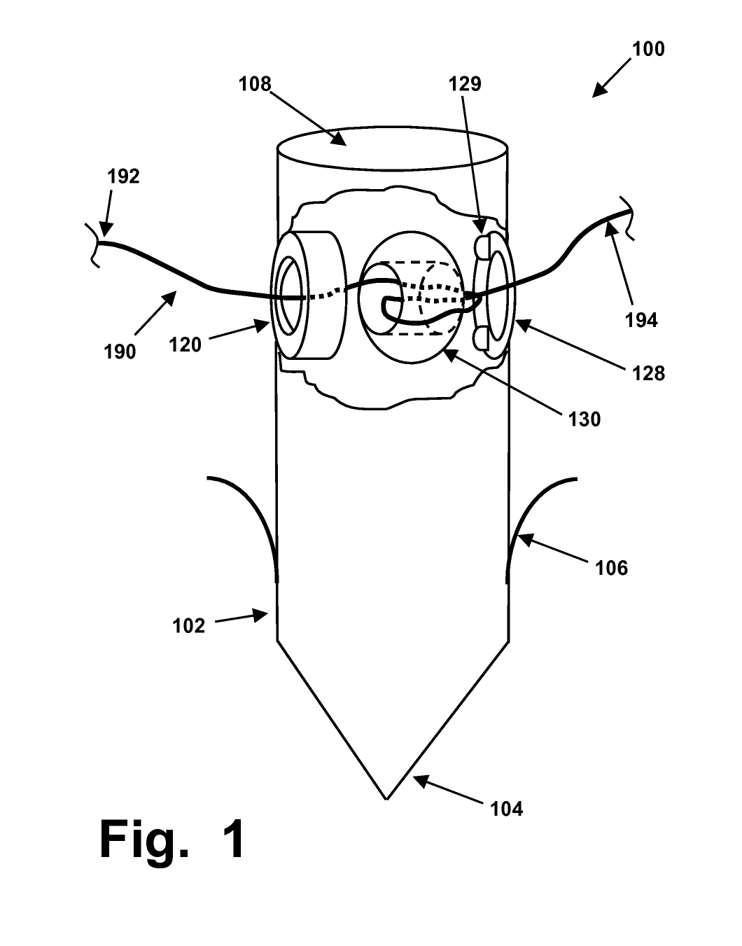

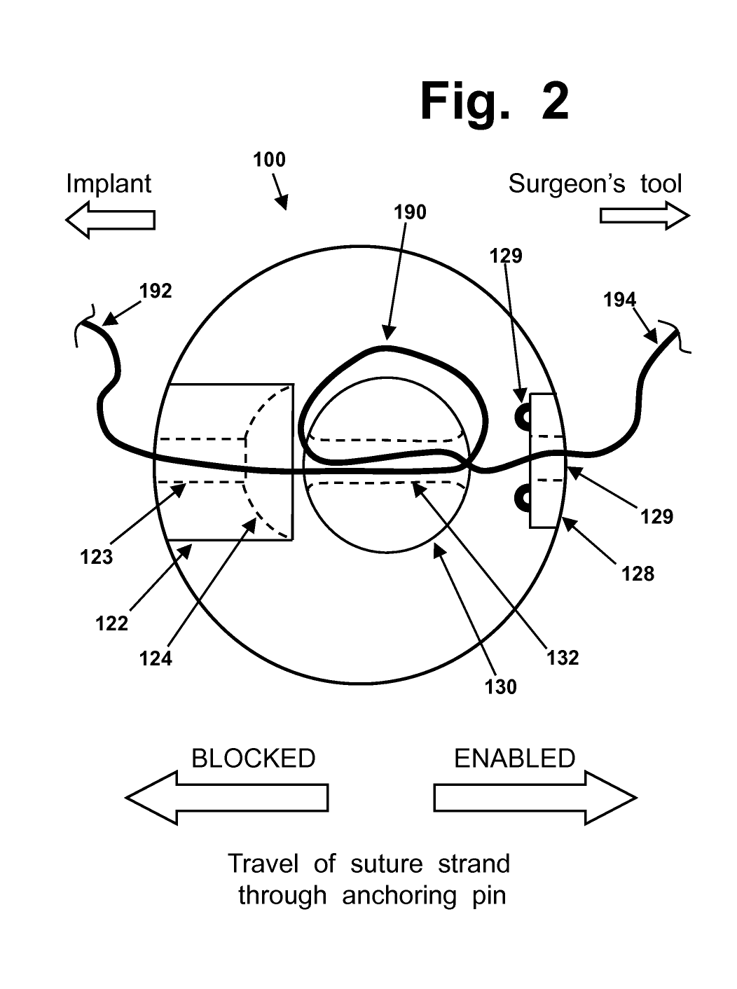

As summarized above, this invention involves knotless suture anchors, which contain an internal mechanism that exerts a ratcheting effect and activity on one or more suture strands that pass through the anchoring device. This type of knotless suture anchor 100 is depicted in FIGS. 1 and 2. To more clearly depict the various non-planar surfaces that will interact with each other, both the cutaway elevation (side) view shown in FIG. 1, and the plan (top) view shown in FIG. 2, should be regarded as semi-schematic or semi-exploded views, in which the separation shown between the components is exaggerated.

Anchor 100 comprises an outer barrel 102, which can also be referred to as a sleeve, enclosure, jacket, outer wall, or similar terms. It can have a cylindrical, square, rectangular, elliptical, or other suitable cross-section. The outer barrel has a tip or end 104, which is suited to be driven into a bone, presumably into a pre-drilled pilot hole. Alternately, for anchors that are designed to be affixed to soft tissues, any other suitable attachment type of attachment means (such as a flap or collar of soft flexible material which can be penetrated by surgical needles or staples) can be provided.

If desired, the outer barrel 102 and/or tip 104 can have external threads, which would allow the anchor to be screwed into a pilot hole in a hard bone. If this type of screw-emplacement design is used, then the anchor will also require:

(1) a recessed socket or comparable accommodation, on the upper surface 108 of anchor 100, to engage the tip of a hex wrench or comparable driving tool; and,

(2) a suitable means for threading one or more suture strands through the anchor device 100, after the anchor device has been screwed into a bone or otherwise emplaced in a targeted tissue. This can be accomplished by emplacing a small segment of a very thin flexible wire, with a loop at one end and a gripping component at the other end, within the ratcheting mechanism of the anchor device and occupying the same looped position that will later be occupied by a suture strand, during manufacture of the device. After the anchor device has been screwed into a bone surface or otherwise emplaced in soft tissue by a surgeon, the surgeon can insert the end of an anchoring or reinforcing suture strand, coupled to an implant device, through the small loop of wire that emerges from one side of the ratcheting anchor device. The opposite end of the wire segment is then gently pulled, by the surgeon, in a manner which will pull the end of the suture strand through the anchor, so that the suture strand will replace the wire segment, which will be discarded after it has been used to pull or “tow” the suture strand into position, within the ratcheting mechanism of the anchor device.

If the outer surface does not have external threads, and is designed to be driven into a bone surface (presumably into a pre-drilled pilot hole) by a tapping or similar operation, then the shaft of the anchoring device preferably should have a plurality of barbs or comparable protrusions 106, to help secure the anchor 100 and prevent it from being pulled out of the bone surface or other tissue.

A generally upper region of anchoring device 100 contains the mechanism or subassembly that will provide the type of ratcheting action and effect that is desired and useful, for handling a suture strand as described herein. The main components of this ratcheting mechanism or subassembly include: (i) a seating component 120 (also called seat 120, for convenience), which will be affixed to a first side of outer barrel 102 anchor 100; (ii) a retainer device 128, which also can be called a washer, grommet, or similar terms, and which will be affixed to a second side or surface of outer barrel 102; and, (iii) a movable component, which is and trapped between the seat 120 and the retainer 128, and which is referred to herein as brake 130 (it can also be called a bead, ball, or similar terms).

As shown more clearly in FIG. 2, the ratcheting subassembly provides:

(i) an entry orifice 123, which passes through seat 122, and which allows suture strand 190 (which will have an implant end 192, coupled to a cartilage-replacing or other implant device, and a free end 194, which can be gripped and pulled by a surgeon's tool) to enter one side surface of anchoring device 100; and, (ii) an exit orifice 129, which passes through retainer component 128, and which allows the free end 194 of suture strand 190 to exit the anchoring device 100. Within the ratchet subassembly (i.e., between entry orifice 123, and exit orifice 129), the suture strand 190 will be wrapped in a single loop, around brake component 130, in a manner as illustrated in FIGS. 1 and 2. This configuration, and the forces exerted on the movable brake or bead 130 by suture strand 190 (which generally will be much more taut and tight than the loose and relaxed configuration shown in FIGS. 1 and 2) will trap the brake or bead 130 in a pathway that allows it to move somewhat, but only in a limited and constrained manner, back and forth between the seat 122 and the retainer 128.

The retainer device 128, which surrounds the strand exit orifice 129, normally will be positioned so that it is diametrically opposed to the seat component 120, which surrounds the strand inlet orifice 123. Alternately, the inlet and exit orifices can be positioned at other angles, if desired, for specialty uses. For example, if an anchoring device of this type will be positioned at the most distant accessible location within an operating field, it may be more convenient for a surgeon to use an anchoring device which has an angle, between the inlet and outlet pathways, somewhere within a range of about 110 degrees up to about 150 degrees. Preferably, if that type of angle is provided, it generally should be an obtuse rather than acute angle, to prevent an acute directional change from interfering with the ratcheting activity and effect of the anchoring device.

The seating surface 124, in seating component 120, is sized and shaped in a manner that accommodates and conforms to the size and shape of the brake or bead 130. Accordingly, brake 130 will settle into, and press firmly against, said seating surface 124, when tension is applied to the implant end 192 of suture strand 190. The seating and pinching action that will arise, when a tensile force tries to pull suture strand 190 toward the implant device, will effectively pinch, trap, grip, and hold the suture strand 129 between the brake or bead 130, and the seating surface 124, in a manner which will block and prevent suture strand 190 from being pulled toward the implant device.

To help ensure an optimal seating interface between movable brake or bead 130, and seating surface 124 of seat component 122, conforming and accommodating surfaces should be provided on: (i) the seating surface 124; and, (ii) the side or surface of the brake or bead 130 that will directly contact and rest against the seating surface 124. If desired, rounded surfaces can be used, with a generally spherical brake or bead, and a generally hemispherical or “rounded saucer” seat. However, round shapes tend to pose a greater risk that a brake or bead of this type might become stuck (or jammed, wedged, lodged, or similar terms) in the seating surface, compared to accommodating conical shapes. Accordingly, unless and until testing indicates otherwise, accommodating conical shapes for the seating surface, and the brake component, are deemed to be somewhat preferable, to spherical and hemispherical shapes. In addition, it also should be noted that the suture strand, passing through the entry and exit orifices and through the bead as well, will help ensure that the brake component will remain properly aligned with the seating component.

The brake or bead component 130 surrounds, encloses, and provides a pathway 132, which also can be called a hole, tunnel, orifice, etc. That pathway can be formed by molding, drilling, or other suitable means, and its edges preferably should be rounded, to prevent abrasion and possible breakage of the suture strand. Its shape and placement will allow suture strand 190 to be:

(1) inserted all the way through the brake, in a left-to-right direction when illustrated as in FIGS. 1 and 2;

(2) looped halfway around the brake, as illustrated in FIGS. 1 and 2; and,

(3) inserted through the brake hole 132 a second time, in the same direction as the first insertion.

This will create a single continuous loop of suture strand material, which will encircle half of the brake or bead 130. In this manner, the suture strand is provided with an ability to directly pull and move the brake in either direction, i.e., either: (1) toward either the seating component 122, shown on the left sides of FIGS. 1 and 2; or, (2) toward the retainer 128, shown on the right sides of FIGS. 1 and 2. However, as soon as brake 130 “bumps into” and presses against either the seating component 122 (if being pulled back toward the implant device), or the retainer 128 (if being pulled away from the implant, by a surgeon using a tool), no further travel of the brake is possible; accordingly, the suture strand can continue to travel, only if the entire segment of strand material (including the loop) is able to slide along any and all surfaces it contacts, including: (1) the internal surface of seating component orifice 123; (2) the internal surface of brake hole 132; (3) the outer surface of brake 130; (4) the internal surface, once again, of brake hole 132; and; the internal surface of the orifice 129, which passes through retainer 128.

In direct contrast to the “snug and conforming” seating interface between brake 130 and seating component 122, one or more small bump- or nodule-type protrusions 129, located on the “retainer side” of brake 130, or on the brake-contacting interior surface of retainer 128, will have the opposite effect, and will prevent brake 130 from pressing against retainer 128 in a snug and/or conforming manner. Therefore, even when brake 130 is being pulled directly into and against retainer 128, by a tensile force exerted on the free end 194 of suture strand 190, the suture strand 190 will NOT become caught, pinched, or otherwise gripped and held by the very small contact points that will be created between brake 130, and the interior surface of retainer 128, if small bumps, nodules, or other protrusions are provided, either on the “retainer side” of the brake component, or on the retainer surface, to prevent the brake from nestling into and resting securely against the retainer.

As a result, the surgeon will be able to pull the suture strand through the anchoring device 100 in one direction, as indicated in FIG. 2, but tension from the implant will not be able to pull the suture strand back through the anchoring device in the opposite direction, toward the implant device. Accordingly, this will generate and provide the type of ratcheting mechanism effect that is desired, in this type of ratcheting knotless suture anchor.

A “rough prototype” of this type of mechanism has been assembled and tested by the Applicant herein, an orthopedic surgeon, using a combination of: (i) braided suture materials made of ultra-high-molecular-weight polyethylene (UHMWPE), which were available to him in his practice as a surgeon; and, (ii) small conical beads and pre-drilled cones (the beads had diameters of about 4 mm, and both the beads and cones were made of unidentified non-rusting metal alloys) that he found at a crafts store. Upon using those components to assemble and test the types of devices shown in FIGS. 1 and 2, and based on having performed hundreds of rotator cuff repairs over a span of decades as a practicing orthopedic surgeon, he discovered that those bead-and-cone assemblies were able to generate gripping forces that were more than strong enough, with an adequate margin of safety, to serve as fully functional and suitable suture-ratcheting devices for use during procedures such as rotator cuff repairs, even when the beads were made of hard and unyielding metal alloys.

Furthermore, even higher and stronger gripping and trapping force will be generated, if somewhat soft and pliable materials (which can be created having any desired level of softness, flexibility, and pliability, using well-known polymer technology) are used to make the movable brake component, and/or the seating surface, in a ratcheting anchor with a design as disclosed herein.

Accordingly, even at that earliest stage of testing, it became clear to the Applicant herein that this approach to designing miniaturized ratcheting suture anchors is entirely feasible and practical.

It also should be noted that different types of strand materials will provide varying levels of “gripping strength”. In general, suture strands made from multiple smaller fibers, either in twisted form (comparable to conventional jute ropes) or braided form (comparable to braided nylon ropes) will provide textured and non-smooth surfaces that can generate higher levels of “gripping strength” (i.e., the ability to resist tensile forces that otherwise would pull a strand in a non-allowed direction), compared to “monofilament” strands (i.e., single-stranded fibers with smooth surfaces, comparable to most types of conventional fishing lines, usually created by forcibly extruding a hot liquefied polymer out of a single extrusion orifice). Accordingly, twisted or braided (rather than monofilament) suture strands are likely to be preferred for most uses described herein. However, any surgeon who has tested and tried out these new types of suture anchors, using various types of candidate suture materials, can develop his/her own preferences for certain types and thicknesses of suture strands, for use in various different types of surgical procedures as described herein.

In addition to pointing out that different types and thicknesses of suture strand materials can be used with these types of racheting anchors (with braided suture materials, which are available in a range of thicknesses, providing a presumptively preferred choice for such uses, subject to the surgeon's personal preferences for any specific patient), it should also be noted that any surface of a movable brake component or a seating component in the types of ratcheting anchors described herein can be provided with a textured, patterned, or other non-smooth surface, or with additional types of barbed, sawtoothed, hooked, “biased bristle”, or other components, positioned at one or more suitable locations within the ratcheting mechanism. For example, the internal tunnel which passes through a movable brake component, and/or one or more edges of such brake component, can be provided with a series of edges shaped in a sawtoothed, gnarled, or other pattern, or with a set of “directionally biased” miniaturized teeth or other protrusions, of the type that are found on the surfaces of various types of rasps that as used to work with wood, metal, or other solid materials. Accordingly, when such additional textures, barbs, hooks, and similar non-smooth surfaces or components are taken into account, it is more accurate to say that at least one seating surface must “engage” at least one surface of a movable brake component, rather than requiring that such “mated” surfaces must “conform” to each other. To be covered by the claims, such “engagement” requires the creation of a racheting effect or activity, when applied to a suture strand (i.e., the strand must be able to travel in a first allowed direction without major impedance, while travel in the opposite direction must be effectively blocked and prevented).

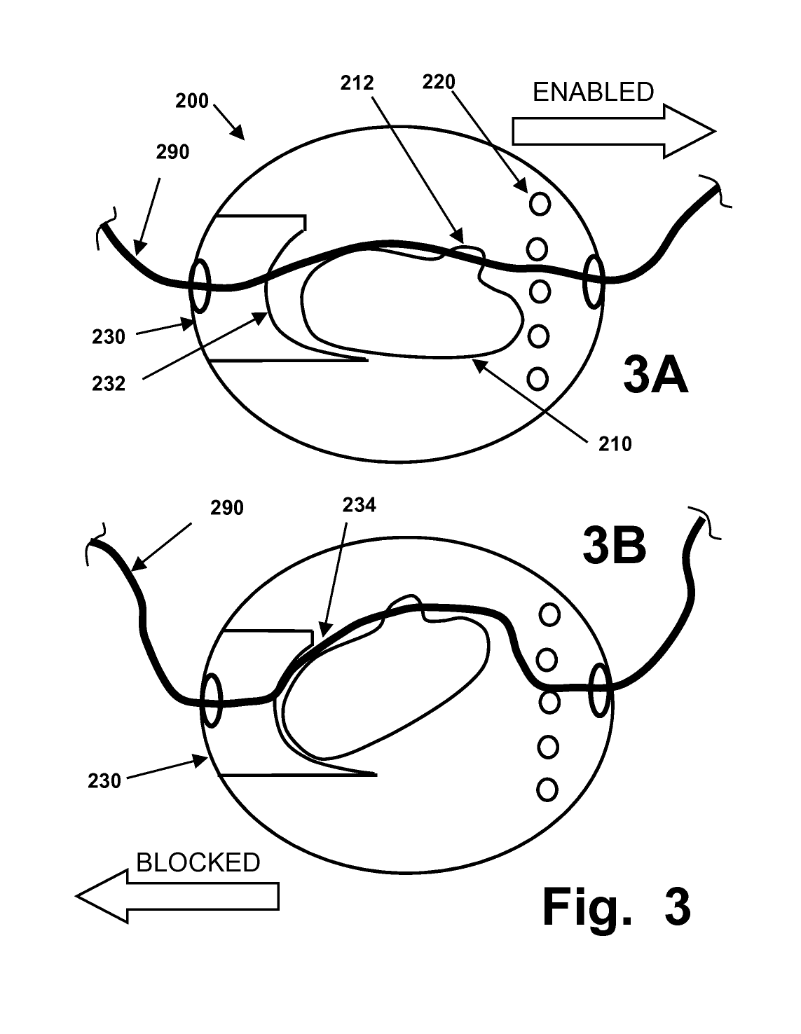

Another design which falls within the class of ratcheting anchor devices covered by the claims is shown in FIG. 3, which includes panels 3A and 3B. In this drawing, ratcheting suture anchor 200 comprises a suture entry orifice 202 and a suture exit orifice 204, and a movable brake component 210 which is slidably coupled to a braided suture strand 290. That coupling involves a side-mounted (rather than centered) eyelet component 212, which has a tunnel passing through it with a diameter that is small enough to exert a steady but yieldable gripping force on a relatively large-diameter suture strand 290. Braided suture strands are well-suited for this type of use, since they have sufficient thickness, and sufficient compressibility (or “yieldability”, or similar terms), to enable a braided strand to engage a small-diameter tunnel in a “gripping but yielding” manner, which will:

(1) allow the suture strand 290 to be pulled through the eyelet 212 of brake component 210 in the “allowed” direction, as indicated in FIG. 3A, without requiring undue force; and,

(2) enable the suture strand to pull the brake component 210 into engagement with the seating component 230, as indicated in FIG. 3B, in a manner which will create a pinching (or gripping, crimping, or similar terms) zone 234, which will grip and trap the braided suture strand 290 between the brake component 210, and the seating surface 232.

FIG. 3 also indicates a retainer structure 220, near the suture exit orifice 204. That retainer structure can have any of various shapes or surfaces, such as a loose mesh, a grid of parallel bars, a molded waffle-type surface, or any other type of surface which will not create the type of enlarged pinching and gripping zone that will be created when the movable brake 210 is pulled against the seating component 230.

Accordingly, when the types of ratcheting mechanisms shown in FIGS. 1-3 are considered, it becomes clear that one of the advantages of these types of racheting mechanisms, compared to the “ring cinch” mechanism shown and described in U.S. application Ser. No. 13/073,342 (Fanton et al; published as 20110238113), is that the ratcheting mechanisms disclosed herein have only a single moving part (this disregards and does not count the suture strand itself, which will simply slide through the mechanism, in a tightly constrained pathway, while being pulled by an external force). By contrast, each of the “cinch” mechanisms described in U.S. application Ser. No. 13/073,342 requires at least two distinct internal moving parts, which can change their positions relative to each other and to their housing assembly. Reducing the number of moving parts by half, within these types of miniaturized devices, leads to three advantages, which can be summarized as follows:

(1) simpler, less expensive, and more reliable manufacture of these types of miniaturized devices;

(2) reduced risk of jamming or malfunction, due to interactions between the internal parts and the housing assembly; and,

(3) reduced risk of jamming or malfunction when used in surgical fields that may contain bone chips or other unwanted particulates, which is of particular concern during various types of orthopedic operations (such as when repairing damaged cartilage, as one example).

The methods required for manufacturing these types of anchors is well within the skill of the art. In general, it is likely to be preferable to “trap” the movable brake (or bead) component, inside the anchoring device, by molding the outer shell (or sleeve, barrel, etc.) in the form of bottom and top components which can be snapped, glued, or otherwise affixed to each other. For example, the bottom component can contain point 104, and the lower surfaces of suture inlet 120 and suture outlet 128. A brake/bead 130, which already has had a suture strand (or a thin placement wire, or comparable emplacement tool) passed through its tunnel 132 a first time, wrapped around the brake, and then passed through the tunnel a second time, as shown in FIG. 2, is then suspended over the bottom component, with both ends of the suture strand resting upon the U-shaped lower portions of the entry and exit holes 123 and 129. A top component can then be lowered down onto the bottom component, while the suture strand and bead remain in place.

Alternately, if desired, the entry and exit holes can both be manufactured as part of a single component, such as either:

(i) a semi-circular bracket which will hold the entry and exit holes at the far ends of the C-shaped bracket; or,

(ii) as part of a top (or cap, or similar terms) component.

In either situation, the two ends of a suture strand (or emplacement tool) which has been affixed to a brake component can be inserted through the entry and exit holes (in an in-to-out direction for both holes), in a manner which will effectively suspend and trap the brake component between the entry and exit holes. The top component, with the suture strand and brake component coupled to it, can then be press-fit into (or glued to, or otherwise affixed to) the bottom component.

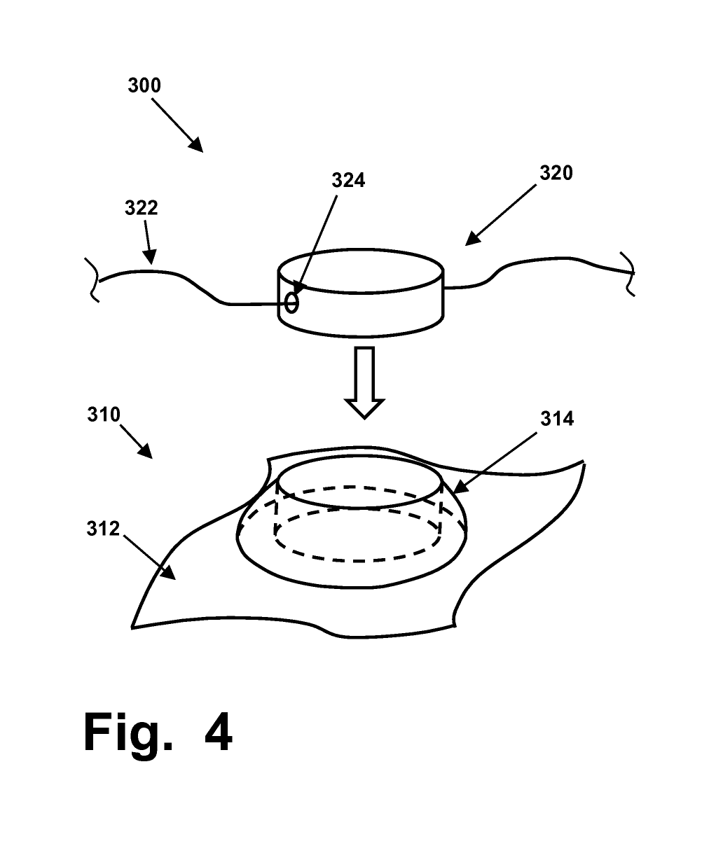

It also should be noted that the anchoring devices disclosed herein can be manufactured in two components, in a manner which will allow the bottom component to be anchored to a bone or soft tissue, within a joint or other surgical site, by itself, before the top component is inserted. Subsequently, the top component, which will carry a movable-brake type of ratchet mechanism with a suture strand affixed to it, can be inserted into the joint or other operating field, and attached to the bottom component. This can render it easier and more convenient for a surgeon to affix a bottom component to a bone surface or soft tissue, without being encumbered by the top component or by a suture strand, and without creating any risk that a miniaturized ratchet mechanism might be damaged by an installation step. This type of two-component anchoring system 300 is illustrated in FIG. 4, which shows:

(1) a lower component 310, with a flexible apron 312 that is designed to be affixed to soft tissue by means of staples, sutures, etc., coupled to a center component 314 that will receive and hold the base of upper component 320; and,

(2) an upper component 320, with a suture strand 322 passing through it (entry orifice 324 is shown; the exit orifice, on the other side, is not shown). Upper component 320 contains a “movable brake” ratchet mechanism as described herein, within the housing component shown in FIG. 4. The upper and lower components can be made of any suitable materials that will enable the upper component 320 to be securely affixed to an already-anchored lower component 310, by any suitable means, such as a snap-ring component, a drop of glue, or a “tab-and-slot” configuration that allows the upper component 320 to be inserted into the lower component 310 and then rotated a fraction of a turn to lock it in place.

It also should be noted that this type of ratcheting knotless anchoring device can be adapted for assembling various types of “implant assemblies”, in an in situ manner (i.e., inside a patient's body, which includes the patients limbs, joints, etc.). As one example, in a shoulder joint, if a rotator cuff has suffered severe damage, or if it became damaged years earlier, and was never repaired in a timely manner, an optimal approach to repairing the damage may involve a sequence of steps in which:

(i) a segment of reinforcing mesh is affixed to an extended edge or area of the torn rotator cuff;

(ii) an accommodating securing device is affixed to the enlarged end of the humerus (i.e., the long bone in the upper arm; and,

(iii) a set of suture strands with ratcheting anchors, as described herein, is used to pull the reinforcing mesh (and the tissue of the torn rotator cuff, in a manner that will pose less risk of tearing, distortion, or other damage of that tissue) to the securing device that has been attached to the bone. In that type of approach, strands or segments of the reinforcing mesh (rather than the suture strands that were used to pull the mesh) can be affixed directly to the securing device that has been anchored to the bone That type of procedure which can be regarded as in situ assembly of an implant device, wherein:

(1) the assembly of that type of implant device will be completed, inside the patient's body, when the reinforcing mesh component has been attached to the securing device; and,

(2) the complete implant, once it has been assembled in situ, has an improved ability to benefit the patient, compared to any other type of implant device that could have been inserted into the patient's body as a sole and single component.

### Sterile Kits Containing Ratcheting Suture Anchors

As will be recognized by anyone who is familiar with how medical devices and surgical implants are manufactured, stored, and shipped, the types of racheting suture anchors disclosed herein can be manufactured, packaged, stored, and shipped in the form of pre-assembled parts and/or subassemblies, commonly known as “kits. To maintain sterility, these types of kits are stored within sealed air-tight and water-tight enclosure (such as relatively thick and durable types of plastic shrink-wrapping), which are designed to be opened only in a sterile operating room or similar environment, immediately prior to use by a surgeon.

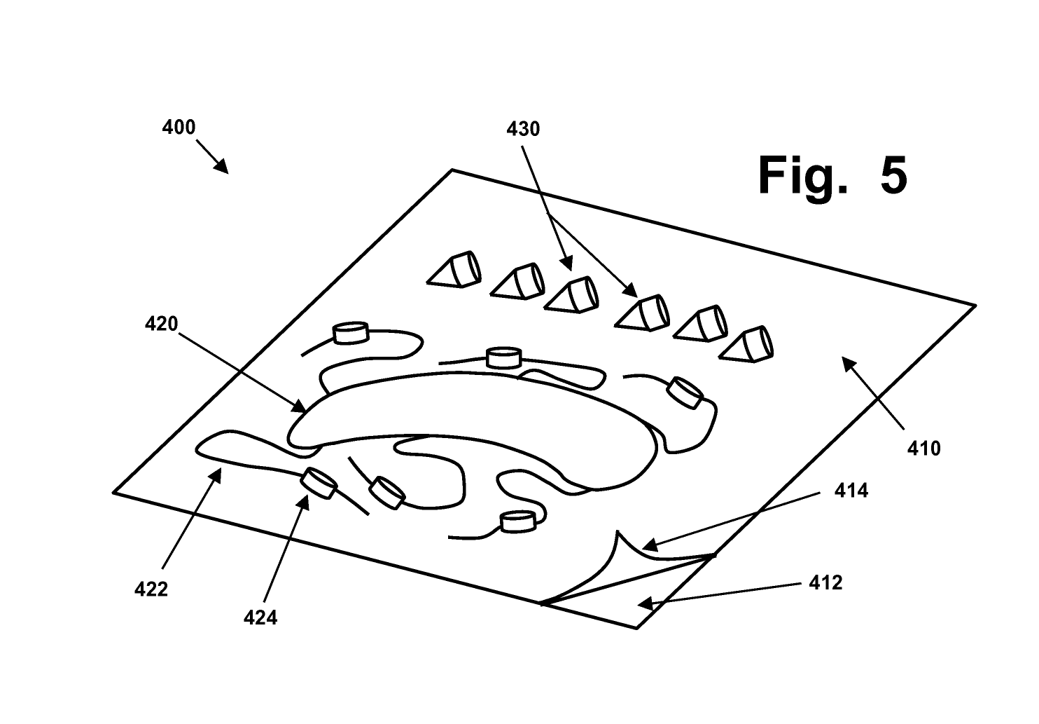

Accordingly, an example of such a kit 400 is provided in FIG. 5, which shows a sealed plastic envelope 410, having a bottom layer 412 and a top layer 414 (shown peeled up at one corner, to illustrate the two distinct layers). Envelope 410 contains, in this example, a cartilage-replacing implant 420, which is sized and shaped for use in replacing the hyaline cartilage on a femoral runner, in a knee joint. This example shows six suture strands 422, each one emerging from the peripheral edge of the femoral implant 420 (for example, each implant can be wrapped around a flexible anchoring cable that is embedded within a molded polymeric component, around the entire periphery of the implant). Each suture strand 422 has a racheting mechanism 424 coupled to it. Each such racheting mechanism is designed to be coupled to an anchoring component 430, after the anchoring component has been driven into a bone surface. The ratcheting mechanisms 424, and the anchoring components 430, are illustrated with exaggerated sizes, to simplify the illustration.

Accordingly, a typical kit can include, for example:

1. one or more fixation devices, which can include either or both types of devices shown in FIGS. 1 and 4, for attaching an anchor to either hard bone and/or soft tissue, and which can be inserted into a joint, body cavity, or other operating site, prior to insertion of a racheting mechanism into the targeted site;

2. one or more brake components, which are designed to be threaded onto suture strands and then inserted into a ratcheting suture anchor as described herein; and,

3. one or more ratcheting mechanisms, which are designed to be inserted into an operating site and then securely affixed to an already-anchored fixation device.

If desired, these types of kits can also include other components and/or devices, such as, for example, cartilage-replacing implants, segments of mesh or other materials that will be used to reinforce a segment of damage tissue that is being repaired, etc.

If desired, additional design features can be used to facilitate in situ assembly of a racheting suture anchor. For example, the lower component (i.e., with fixation/anchoring means that will allow the lower component to be driven into a bone surface, or stapled or sutured to soft tissue) can be provided with suture entry and exit components with crimped access slots on their top edges. After a surgeon has threaded a brake component onto a suture strand, the surgeon can use conventional arthroscopic or laparoscopic tools to:

(1) press the suture strand into the entry orifice, in a manner which will use the crimped orifice to secure the suture strand in that location;

(2) adjust the positioning of the suture strand, until the brake component (which can slide along the length of the suture strand, but only with resistance) is positioned between the entry and exit orifices;

(3) press the suture strand down into the crimped exit orifice, to ensure that the suture strand and the brake component will not move out of position as the ratchet mechanism is being inserted into the operating area and then affixed to the anchoring component.

### Handling Long Threads, and Preserving a Natural “Feel”

All of the ratchet mechanisms disclosed herein will allow a suture strand to pass entirely through an anchor device, in a manner which will provide a “free end” of the suture strand. This can provide two potentially important benefits, compared to the type of rotating ratchet mechanism disclosed in the published applications by Van der Burg et al.

First, the “direct pass-through” nature of the ratchet mechanisms disclosed herein will allow substantially longer suture strands to be used, for initial anchoring and preliminary reinforcing of a cartilage-replacing implant device, compared to a rotating ratchet system which would need to stuff long suture strands inside miniaturized cylinders that are kept as small as possible. The types of flexible cartilage-replacing implant devices being developed by the Applicant herein will pose substantial challenges, especially to surgeons must learn how to use them effectively. A set of relatively long and easily-reachable suture strands, positioned at key locations around the periphery of an implant, will effectively provide a set of “handles” that can be used by a surgeon to help the surgeon manipulate and position an implant, inside a joint which is being manipulated with only limited arthroscopic access.

It should also be noted that the suture strands which are coupled to an implant can be color-coded, to provide a surgeon with an additional set of visual cues, to help the surgeon complete the surgical procedure quickly and effectively.

Secondly, since all of the ratchet mechanisms disclosed herein will allow the “free end” of a suture strand to be grasped and pulled by the surgeon, these designs can preserve a normal and natural “feel”, which most arthroscopic surgeons would prefer to have, during a tightening procedure. By contrast, a rotating ratchet mechanism, as disclosed in published application 2010/0063542 (Van der Burg et al) will need to be driven by some type of wrench or similar powered tool. Furthermore, in a rotating ratchet system which must be driven by a powered wrench, there is some level of risk that the accumulation of a significant length of suture strand, in a narrow and tightly constrained gap between an internal rotating device and a surrounding sleeve, might cause the rotation and responsiveness of the mechanism to be altered, and distorted, in ways that cannot be fully predicted or controlled if a substantial length of suture strand is involved.

Finally, it should be noted that the relatively simple “direct pass-through” nature of the ratchet mechanisms disclosed herein can enable various designs and methods for momentarily releasing the grip of a ratchet mechanism on a suture strand, in a way that will allow the tension in the strand to be reduced, if necessary. As just one example, a small sleeve made of smooth-surfaced plastic, with a slit passing through it lengthwise, can be fitted onto the surface of a suture strand, immediately “above” a ratchet mechanism. If the smooth sleeve is pushed into the ratchet mechanism, it can create enough separation, between two gears or similar devices, to enable a suture strand to be pulled backward through the ratchet mechanism.

Thus, there has been shown and described (i) a new and useful class of knotless suture anchors with ratcheting capability, and (ii) a new and useful class of surgical implant devices which incorporate such ratcheting suture anchors. Although this invention has been exemplified for purposes of illustration and description by reference to certain specific embodiments, it will be apparent to those skilled in the art that various modifications, alterations, and equivalents of the illustrated examples are possible. Any such changes which derive directly from the teachings herein, and which do not depart from the spirit and scope of the invention, are deemed to be covered by this invention.

1. An anchoring device for knotless securing of suture strands, wherein said device comprises:
a. at least one fixation component, which is sized and suited for securing the anchoring device to at least one type of bone or internal tissue; and,
b. at least one ratchet-type mechanism which is designed and suited to allow a suture strand to be pulled through said anchoring device in one direction, but which will prevent travel of said suture strand through said anchoring device in an opposing direction;
wherein said ratchet-type mechanism comprises:
(1) a seating component, having a seating surface with a size and shape that will engage at least one surface of a movable brake component when said movable brake component is pressed against said seating component; and,
(2) a movable brake component, which can be slidably affixed to a suture strand, and which has a seat-engaging surface that is sized and shaped to engage the seating surface of the seating component,
and wherein said anchoring device also comprises:
(i) retaining means, which are designed and suited to prevent said movable brake component from disengaging from the anchoring device; and,
(ii) entry and exit means which enable a suture strand to pass through the anchoring device while providing at least one free suture end which can be gripped and pulled by a surgeons tool,
and wherein said movable brake component and said seating component will impose a pinching force on a suture strand which passes through said anchoring device, if a tensile force exerted on said suture strand pulls said movable brake component into compressive engagement with said seating component.

2. The anchoring device of claim 1, wherein said fixation component and said ratchet-type mechanism are separable components which are designed and suited to allow:
a. surgical implantation of said fixation component without said ratchet-type mechanism; and,
b. insertion of said ratchet-type mechanism into an operating site where said fixation component has been affixed to bone or tissue; and,
c. attachment of said ratchet-type mechanism to said fixation component.

3. A suture anchor, suited for surgical use in humans or animals, comprising:
a. means for affixing said suture anchor to at least one type of tissue; and,
b. a racheting mechanism which will allow a suture strand to pass through said mechanism in a manner which allows at least one free end of said suture strand to be gripped by a surgeon's tool, and which will allow said suture strand to pass through said mechanism in a first allowed direction when said free end is gripped and pulled, and which will prevent movement of said suture strand in an oppositional direction when said suture strand is pulled by tensile forces that are conventionally imposed on suture strands during orthopedic surgery,
and wherein said ratcheting mechanism comprises:
(1) a seating component, having a seating surface with a size and shape that will engage at least one surface of a movable brake component when said movable brake component is pressed against said seating component; and,
(2) a movable brake component, which can be slidably affixed to a suture strand, and which has a seat-engaging surface that is sized and shaped to engage the seating surface of the seating component.

5. A surgical kit, comprising components for assembling a racheting suture anchor within a sealed plastic enclosure designed to ensure sterility of said components, wherein said components include:
a. at least one subassembly which is sized and suited for securing a suture anchor device to at least one type of bone or internal tissue; and,
b. at least one subassembly which comprises a ratchet-type mechanism designed and suited to allow a suture strand to be pulled through said ratchet-type mechanism in one direction, but which will prevent travel of said suture strand through said ratchet-type mechanism in an opposing direction;
wherein said ratchet-type mechanism comprises:
(1) a seating component, having a seating surface with a size and shape that conform to at least one surface of a movable brake component; and,
(2) a movable brake component, which can be slidably affixed to a suture strand, and which has a seat-engaging surface that is sized and shaped to conform to the grating surface of the seating component.
